(19) United States
(12) Patent Application Publication
(10) Pub. No.: US 2019/0198145 A1
(43) Pub. Date: Jun. 27, 2019

(54) INTERACTIVE PRECISION HEALTH CARE SYSTEM RELATED TO HIGH BLOOD SUGAR

(71) Applicants: TZUNG-SHIARN PAN, Kaohsiung (TW); HUANG-CHUNG TSENG, Pingtung County (TW)

(72) Inventors: TZUNG-SHIARN PAN, Kaohsiung (TW); HUANG-CHUNG TSENG, Pingtung County (TW)

(21) Appl. No.: 15/870,244

(22) Filed: Jan. 12, 2018

(30) Foreign Application Priority Data
Dec. 25, 2017 (TW) 106145543

(51) Int. Cl. G16H 20/10; G16H 15/00

(52) U.S. Cl. CPC G16H 20/10; G16H 15/00

(57) ABSTRACT

An interactive precision health care system related to high blood sugar includes an examination module generating an examination report, a database and a processing module. The database stores the examination report, a characteristic information, and a health care information. The characteristic information has a first range. The processing module receives and compares the examination report with the characteristic information. When a pre-meal blood sugar level falls within the first range, the processing module generates a dietary supplement product list including a product information and a time schedule of taking medicine. The processing module controls a delivery module to proceed with delivery. The processing module controls a reminding module to send out a message of taking medicine to obtain a new examination report. The processing module modifies the dietary supplement product list. The processing module controls the delivery module to proceed with delivery based on the modified dietary supplement product list.

### CROSS REFERENCE TO RELATED APPLICATION

The application claims the benefit of Taiwan application serial No. 106145543, filed on Dec. 25, 2017, and the entire contents of which are incorporated herein by reference.

### BACKGROUND OF THE INVENTION

### 1. Field of the Invention

The present invention relates to an interactive precision health care system related to high blood sugar and, more particularly, to an interactive precision health care system related to high blood sugar for preventing diabetes or delaying the onset of diabetes.

### 2. Description of the Related Art

Diabetes is a chronic disease resulting from abnormal metabolism of carbohydrate. Due to the change in the dietary patterns of people, the global morbidity rate of diabetes is continuously increasing at a rate of 8-10% per year.

Conventional methods for testing diabetes can analyze whether a patient has diabetes through detection of the sugar level in the blood by blood tests. However, the abnormal blood sugar level in the human body cannot be detected without intentional examination. Due to irreversibility of diabetes, the patient generally has to take medicine for controlling the blood sugar for the rest of his or her life after confirmation, which is too late.

Furthermore, some people rely on dietary supplements to prevent diabetes, yet the types of dietary supplements cannot be adjusted according to personal body conditions.

Thus, a need exists for a health care system for preventing the occurrence of diabetes.

### SUMMARY OF THE INVENTION

To solve the above problems, the present invention provides an interactive precision health care system related to high blood sugar for preventing diabetes or delaying the onset of diabetes.

An interactive precision health care system related to high blood sugar includes an examination module, a database and a processing module. The examination module is configured to generate an examination report. The database is coupled to the examination module and is configured to store the examination report, a characteristic information, and a health care information. The characteristic information has a first range. The processing module is coupled to the database and is configured to receive and compare the examination report with the characteristic information. When a pre-meal blood sugar level falls within the first range, the processing module generates a dietary supplement product list based on the examination report and the health care information. The dietary supplement product list includes a product information and a time schedule of taking medicine. The processing module stores the examination report and the dietary supplement product list in the database and controls a delivery module to proceed with delivery according to the dietary supplement product list. The processing module controls a reminding module to send out a message of taking medicine based on the time schedule of taking medicine of the dietary supplement product list to obtain a new examination report. The processing module modifies the dietary supplement product list based on the new examination report as well as the examination report and the dietary supplement product list of a same user of the new examination report in the database. The processing module stores the new examination report and the modified dietary supplement product list in the database and controls the delivery module to proceed with delivery based on the modified dietary supplement product list.

In view of the foregoing, the interactive precision health care system related to high blood sugar according to the invention can immediately adjust the types of the supplement products according to the blood sugar value, the physiological manifestation, and the reactions after taking the supplement products. Thus, those people who are liable to have diabetes can be aware of their risks. Therefore, their high blood sugar can be reversed based on the self-adjustment mechanism of the human body in early stage to bring the blood sugar back into the normal range, thus preventing or delaying the onset of diabetes.

In an example, the processing module generates the dietary supplement product list based on whether the examination report includes a symptom of diabetes. When the examination report does not include the symptom of diabetes, the processing module generates a dietary supplement product list including a health care information of preventing diabetes. When the examination report includes the symptom of diabetes, the processing module generates a dietary supplement product list including a health care information of controlling diabetes. Thus, it is possible to prevent and control diabetes at the same time.

In an example, the processing module controls the reminding module to send a reply message to obtain the new examination report. This prevents the user from forgetting to send the new examination report, increasing the accuracy of the dietary supplement product list.

In an example, the processing module generates and sends a reexamination signal to restart obtaining the examination report when the pre-meal blood sugar level is outside of the first range. Thus, the patients with pseudo diabetes can be excluded from medical treatment, avoiding the waste of medical source.

In an example, the examination module includes a measuring module and a human machine interface. The measuring module is configured to generate a blood sugar report. The human machine interface is configured to input a user feedback report.

In an example, the human machine interface is configured to obtain a questionnaire from the database, and the user feedback report is generated by using the questionnaire. Thus, the answers of the user can be standardized to further improve the accuracy of the dietary supplement product list.

In an example, the interactive precision health care system related to high blood sugar further includes an inquiry end coupled to the processing module. Thus, the on-line inquiry can be conducted to provide the privacy of use.

In an example, the interactive precision health care system related to high blood sugar further comprises a login module coupled to the processing module. This can verify the identification of the user, thus providing precise medication.

The present invention will become clearer in light of the following detailed description of illustrative embodiments of this invention described in connection with the drawings.

### BRIEF DESCRIPTION OF THE DRAWINGS

FIG. 1 shows a block diagram of an interactive precision health care system related to high blood sugar.

### DETAILED DESCRIPTION OF THE INVENTION

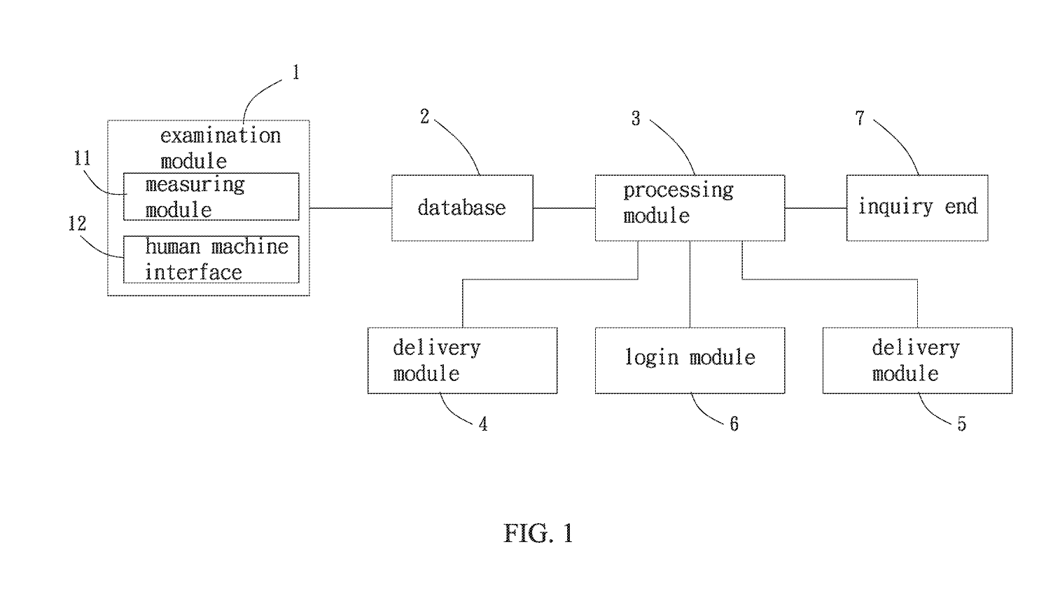

FIG. 1 shows an interactive precision health care system related to high blood sugar according to a preferred embodiment of the invention. The interactive precision health care system includes an examination module 1, a database 2, a processing module 3, a delivery module 4 and a reminding module 5. The database 2 is coupled with the examination module 1. The processing module 3 is coupled with the database 2. The delivery module 4 and the reminding module 5 are coupled with the processing module 3.

The examination module 1 is configured to generate an examination report including a blood sugar report and a user feedback report. In this embodiment, the examination module 1 includes a measuring module 11 and a human machine interface 12. The measuring module 11 may be any module configured to measure the blood sugar value. The measuring module 11 is configured to measure a pre-meal blood sugar level and a post-meal blood sugar level, so as to generate the blood sugar report. Preferably, the measuring module 11 is a non-invasive blood sugar meter. The human machine interface 12 may be a device with an input function, an output function and a display function. The human machine interface 12 is configured to receive the user feedback report from the user. The user feedback report includes at least one question regarding whether the user has one or more symptoms of diabetes such as increased appetite and urination.

The database 2 may be any storage module configured to store data. The database 2 is coupled with the examination module 1 for storing the examination report. Preferably, when the database 2 is a cloud server, the examination module 1 can have a wireless transmission function to transmit the examination report to the database of the cloud server. Furthermore, the database 2 can store a characteristic information and a health care information. Specifically, the characteristic information may include a first range and a second range. In this embodiment, the first range is a pre-meal blood sugar range. Namely, the pre-meal blood sugar range on a 12-hour fasting is between 110-126 mg/dl. The second range is a post-meal blood sugar range. Namely, the post-meal blood sugar range measured 1 hour after the meal is between 150-200 mg/dl. The health care information is a product information and can include at least one of a dietary information and an exercise information. The product information preferably includes two types of products. One of the two types of products is associated with the products for preventing diabetes, and another one is associated with the products for controlling diabetes. In a non-limiting example, the products for preventing diabetes can include wild balsam pera, chromium yeast and cinnamon, and the products for controlling diabetes can include wild balsam pera, chromium yeast, cinnamon and salvia miltiorrhiza extract. However, this is not used to limit the invention.

The processing module 3 is coupled with the database 2 to obtain the examination report from the database 2. The processing module 3 compares the examination report with the characteristic information. If the blood sugar report of the examination report reveals that the pre-meal blood sugar level does not fall within the first range, the processing module 3 transmits a reexamination signal regardless whether the post-meal blood sugar level falls within the second range. This permits the measuring module 11 of the examination module 1 to reexamine the pre-meal blood sugar level and the post-meal blood sugar level of the user after a period of time, such that a new examination report can be generated and transmitted to the database 2. If the blood sugar report of the examination report reveals that the pre-meal blood sugar level falls within the first range, the processing module 3 generates a dietary supplement product list based on the examination report and the health care information. The dietary supplement product list is customized and suitable for the user. The processing module 3 sends the dietary supplement product list to the user for the user's confirmation. The dietary supplement product list includes at least a product information and a time schedule of taking medicine.

The processing module 3 can generate the dietary supplement product list according to whether the user feedback report contains the symptoms of diabetes. Specifically, if the user feedback report does not contain the symptoms of diabetes, the processing module 3 generates a dietary supplement product list associated with preventing diabetes. On the contrary, if the user feedback report contains the symptoms of diabetes, the processing module 3 generates a dietary supplement product list associated with controlling diabetes.

Subsequently, the user reviews the dietary supplement product list and sends a response signal to the processing module 3. If the response signal is an acceptance signal, the processing module 3 sends the dietary supplement product list to the delivery module 4. The delivery module 4 sends the corresponding dietary supplement products to the user based on the dietary supplement product list by ways of posting, hand delivery or delivery service, for example. However, this is not used to limit the invention. If the response signal is a non-acceptance signal, the processing module 3 can adjust the dietary supplement product list. The adjusted dietary supplement product list is sent to the user for re-confirmation. Once the user agrees with the dietary supplement products of the dietary supplement product list, the processing module 3 stores the dietary supplement product list in the database 2 for future consideration when making an adjustment to the dietary supplement product list. Preferably, the user feedback report may include the information regarding other medicines the user is currently taking. This information can also be taken into consideration when making an adjustment to the dietary supplement product list.

Furthermore, the processing module 3 reminds the user to take the medicine by controlling the reminding module 5 to send out a message of taking medicine based on the time schedule of taking medicine of the dietary supplement product list. After the user takes the medicine, the examination module 1 generates and sends a new examination report to the database 2. Accordingly, the processing module 3 modifies the dietary supplement product list based on the new examination report of the user as well as the examination report and the dietary supplement product list which are stored in the database 2 and belonged to the same user. The processing module 3 sends the modified dietary supplement product list to the user for review. Once the processing module 3 receives the acceptance signal, the processing module 3 controls the delivery module 4 to deliver the related products to the user based on the modified dietary supplement product list, and stores the new examination report and the modified dietary supplement product list in the database 2. Thus, precise medication can be attained. Moreover, after the user takes the medicine, the user may forget to use the examination module 1 to produce and transmit a new examination report to the database 2. To prevent this from happening, the processing module 3 can remind the user to do so by controlling the reminding module 5 to transmit a reminding message to the user after a period of time. In this embodiment, the reminding message is transmitted once a week, but is not limited thereto.

The interactive precision health care system according to the invention may further include a login module 6 coupled with the database 2. The user can log in the login module 6 with the accounts of the social networks such as Facebook, Instagram, Twitter or Line. Alternatively, the user can register at the social networks by using the login module 6. However, this is not used to limit the invention.

The user feedback report can be produced by surveying or on-line inquiry. In the case of surveying, the database 2 can store a questionnaire in advance. Then, the user receives and fills out the questionnaire. The completed questionnaire is used as the user feedback report and sent to the processing module 3 for analysis. In the case of on-line inquiry, the interactive precision health care system according to the invention may include at least one inquiry end 7 coupled with the database 2. The inquirer at the inquiry end 7 can directly inquire the user on line and can use the human machine interface 12 to generate the user feedback report.

The user can transmit the response signal and receive the reexamination signal, the message of taking medicine and the dietary supplement product list through a device having an input function, an output function and a display function. For example, the device may be a handset, a tablet or a personal computer. The device may also be an input medium of the interactive precision health care system, such as a mouse, a keyboard or a touch panel. Preferably, the interactive precision health care system may further include an audio and video capture device which may be, but not limited to, a camera and a microphone. Therefore, the user can capture their images and voices via the audio and video capture device and communicate with the inquirer on line via the audio and video capture device, improving the privacy of use.

During the use of the interactive precision health care system, the user can log in the interactive precision health care system of the invention through the human machine interface 12 or a handset. The account to log in the interactive precision health care system may be a personal account of a social network. After login, the user can choose to fill out the questionnaire or proceed with the on-line inquiry, so as to generate the user feedback report. In this embodiment, the user fills out the questionnaire to generate the user feedback report. The user uses the measuring module 11 to generate a blood sugar report and acquires a questionnaire by accessing the database 2 with the measuring module 11, thereby generating a user feedback report. The user sends the blood sugar report and the user feedback report to the database 2. The processing module 3 compares the blood sugar report with the characteristic information and generates a dietary supplement product list which may fit the need of the user. The processing module 3 sends the dietary supplement product list to the handset of the user for confirmation. If the user agrees with the supplement products in the list, the user sends an acceptance signal to the processing module 3 via the handset. Then, the processing module 3 controls the delivery module 4 to deliver the supplement products to the user according to the dietary supplement product list. To remind the user to take the medicine, the processing module 3 controls the reminding module 5 to send a message of taking medicine to the handset of the user according to the time schedule of taking medicine. After the user takes the medicine, the processing module 3 sends a reply message to the user, requesting the user to send a new blood sugar report and a new user feedback report to the database 2 through the measuring module 11 and the human machine interface 12. In this regard, the processing module 3 re-determines whether it is needed to adjust the dietary supplement product list of the user according to the new blood sugar report, the new user feedback report, the previous blood sugar report, the previous user feedback report, as well as the dietary supplement product list. If the determined result is positive, the processing module 3 re-adjusts the dietary supplement product list in a manner more suitable for the current condition of the user and controls the delivery module 4 to deliver the supplement products to the user. If the determined result is negative, the current dietary supplement product list is used to provide the current supplement products to the user.

In view of the foregoing, the interactive precision health care system related to high blood sugar according to the invention can immediately adjust the types of the supplement products according to the blood sugar value, the physiological manifestation, and the reactions after taking the supplement products. Thus, those people who are liable to have diabetes can be aware of their risks. Therefore, their high blood sugar can be reversed based on the self-adjustment mechanism of the human body in early stage to bring the blood sugar back into the normal range, thus preventing or delaying the onset of diabetes.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. An interactive precision health care system related to high blood sugar, comprising:
an examination module configured to generate an examination report;
a database coupled to the examination module, wherein the database is configured to store the examination report, a characteristic information, and a health care information, wherein the characteristic information has a first range; and
a processing module coupled to the database, wherein the processing module is configured to receive and compare the examination report with the characteristic information,
wherein when a pre-meal blood sugar level falls within the first range, the processing module generates a dietary supplement product list based on the examination report and the health care information, wherein the dietary supplement product list includes a product information and a time schedule of taking medicine, wherein the processing module stores the examination report and the dietary supplement product list in the database and controls a delivery module to proceed with delivery according to the dietary supplement product list, wherein the processing module controls a reminding module to send out a message of taking medicine based on the time schedule of taking medicine of the dietary supplement product list to obtain a new examination report,
wherein the processing module modifies the dietary supplement product list based on the new examination report as well as the examination report and the dietary supplement product list of a same user of the new examination report in the database,
wherein the processing module stores the new examination report and the modified dietary supplement product list in the database and controls the delivery module to proceed with delivery based on the modified dietary supplement product list.

2. The interactive precision health care system related to high blood sugar as claimed in claim 1, wherein the processing module generates the dietary supplement product list based on whether the examination report includes a symptom of diabetes, wherein when the examination report does not include the symptom of diabetes, the processing module generates a dietary supplement product list including a health care information of preventing diabetes, wherein when the examination report includes the symptom of diabetes, the processing module generates a dietary supplement product list including a health care information of controlling diabetes.

3. The interactive precision health care system related to high blood sugar as claimed in claim 1, wherein the processing module controls the reminding module to send a reply message to obtain the new examination report.

4. The interactive precision health care system related to high blood sugar as claimed in claim 1, wherein the processing module generates and sends a reexamination signal to restart obtaining the examination report when the pre-meal blood sugar level is outside of the first range.

5. The interactive precision health care system related to high blood sugar as claimed in claim 1, wherein the examination module includes a measuring module and a human machine interface, wherein the measuring module is configured to generate a blood sugar report, and wherein the human machine interface is configured to input a user feedback report.

6. The interactive precision health care system related to high blood sugar as claimed in claim 5, wherein the human machine interface is configured to obtain a questionnaire from the database, and wherein the user feedback report is generated by using the questionnaire.

7. The interactive precision health care system related to high blood sugar as claimed in claim 5, further comprising an inquiry end coupled to the processing module.

8. The interactive precision health care system related to high blood sugar as claimed in claim 1, further comprising a login module coupled to the processing module.
